(19) United States
(12) Patent Application Publication
(10) Pub. No.: US 2014/0107698 A1
(43) Pub. Date: Apr. 17, 2014

(54) GASTRIC TRACTION DEVICE AND METHOD

(71) Applicant: Children's Hospital Medical Center, Cincinnati, OH (US)

(72) Inventor: Thomas Inge, Cincinnati, OH (US)

(73) Assignee: Children's Hospital Medical Center, Cincinnati, OH (US)

(21) Appl. No.: 14/046,887

(22) Filed: Oct. 4, 2013

Related U.S. Application Data
(60) Provisional application No. 61/709,444, filed on Oct. 4, 2012.

(51) Int. Cl. A61B 17/072; A61B 17/064

(52) U.S. Cl. CPC A61B 17/072; A61B 17/064
USPC 606/219; 227/175.1

(57) ABSTRACT

A surgical clamp or gastric traction device and method for grasping and manipulating the stomach and providing continuous and uniform tension along a predetermined staple line during the serial stapling and stomach transection portions of a laparoscopic gastric sleeve procedure. The clamp typically includes a handle for gripping and manipulating the clamp and the engaged stomach, and a pair of elongated jaws coupled to the handle via a central portion. The pair of elongated jaws include a first jaw member and a second jaw member, and the length of the jaws are substantially equal to the length of a predetermined staple line which spans the stomach from the pyloric area to the area of the Angle of His. In one embodiment, the handle assembly can impart articulating and pivoting motion to the clamp assembly.

### CROSS-REFERENCE TO RELATED APPLICATION

This application claims priority to U.S. provisional patent application Ser. No. 61/709,444, filed Oct. 4, 2012, the disclosure of which is incorporated herein by reference.

### BACKGROUND OF THE INVENTION

The present invention relates in general to surgical instruments and methods for performing laparoscopic, endoscopic, and traditional open surgical procedures, and in particular to a clamp or gastric traction device for use in laparoscopic sleeve gastrectomy procedures.

Approximately two-thirds of adults in the U.S. are either overweight or obese, and about 5 percent of the population (or 1 in 20 adults) is considered morbidly obese. Obesity rates are categorized by Body Mass Index, or BMI, which is determined by the ratio of a person's height to weight. A person is considered normal with a BMI of less than 25, overweight between 25 and 30 and obese at 30 and higher. Among the latter classification, a BMI of 35 to 39 is considered severe obesity and 40 or higher is morbid obesity. The health consequences of obesity are many, and include an increased risk of premature death from high blood pressure, heart disease, diabetes, obstructive sleep apnea, hypoventilation, degenerative arthritis, and psychosocial impairment. Current evidence suggests surgical therapies offer the best hope for significant and sustainable weight loss in the morbidly obese, with resultant mortality reduction.

Sleeve gastrectomy is a type of restrictive weight loss surgery, recommended only for the morbidly obese, in which the left side of the stomach is surgically removed, thereby reducing the size of the stomach by 60-85%, to provide increased satiety and decreased appetite. Candidates include those with a BMI above 40, or a BMI above 35 with associated obesity-related health conditions such as type II diabetes, hypertension, sleep apnea, severe arthritis, asthma, hypercholesterolemia and cardiovascular diseases. The term “sleeve” refers to the new look of the stomach pouch, which is shaped like a banana or the sleeve of a long sleeved shirt. The reduction is caused by the stapling and removal of the majority of the stomach to create a new, smaller stomach pouch. Sleeve gastrectomy combines the principles of gastric restriction and hormonal appetite suppression by removing the fundus of the stomach and thus the majority of the oxyntic glands that produce appetite stimulating ghrelin and other hormones. Because there is no intestinal bypass associated with this procedure, it is considered safer than other treatment options. However, this form of weight loss surgery is not reversible.

During a minimally invasive, laparoscopic surgical procedure such as a sleeve gastrectomy, access to the surgical site within the body cavity is provided using up to 6 small diameter openings made in the abdominal wall. An instrument typically used to provide access is a trocar, which includes an obturator and a cannula. The obturator has a sharp tip which is used to puncture the body wall to provide the access opening. The obturator slides within the cannula, which is a hollow, cylindrical sleeve. The obturator is removed from the cannula after it has punctured the body wall, and the cannula remains in place as a passageway for inserting various surgical devices such as scissors, dissectors, graspers, retractors, or similar instruments into the body cavity.

To facilitate operability through the cannula, instruments adapted for laparoscopic surgery typically include a relatively narrow shaft supporting an end effector at its distal end and a handle at its proximal end. Arranging the shaft of such an instrument through the cannula allows a surgeon to manipulate the handle from outside the body to cause the end effector to carry out a surgical procedure at a remote internal surgical site. This type of laparoscopic procedure has proven beneficial over traditional open surgery due to reduced trauma, improved healing and other attendant advantages. Accordingly, a characteristic feature of many endoscopic surgical instruments is a long cylindrical shaft which can slide through the cannula of a trocar port. Also, as described in more detail below, single incision laparoscopic surgery (SILS™) is a recent advancement in minimally invasive surgical techniques, and decreases the number of skin incisions to one or two.

In addition to causing volume restriction, sleeve gastrectomy removes a large volume of glandular stomach that affects appetite. The procedure typically includes placing a 12-mm optical trocar under direct vision in the periumbilical area, approximately 15 cm below the xiphoid and 3 cm to the left of midline. A 30- or 45-degree angled laparoscope is then placed through the port into the peritoneal cavity and second 12-mm port is placed in the left lateral flank, medial to the edge of the colon with the patient in a supine position and at the same level as the periumbilical port. Next, a 5-mm trocar port is placed along the left subcostal margin between the xiphoid process and the left flank port. Another 12-mm port is placed in the right epigastria region, and a fourth 12 mm port is placed in the mid-epigastric region caudal and medial to the previous port. The liver is elevated and this provides adequate visualization of the entire stomach during the gastrectomy. The pylorus of the stomach is then identified and the greater curve of the stomach is elevated. An ultrasonic scalpel is then used to enter the greater sac via division of the greater omentum. The greater curvature of the stomach is then dissected free from the omentum and the short gastric blood vessels using the laparoscopic ultrasonic scalpel. The dissection is started 5 cm from the pylorus and proceeds to the Angle of His.

A gastroscope or bougie (i.e. a dilator, or sizing tube) with a 9-14 mm diameter, or between 32-60 French, is then passed under direct vision through the esophagus, stomach, and into the first portion of the duodenum. The bougie is then aligned along the lesser curvature of the stomach and typically used as a template to create the vertical sleeve. An endoscopic linear cutting stapler is then used to serially staple and transect the stomach, staying just to the anatomic left and lateral to the bougie. Stapling proceeds in an upward direction, beginning near the antrum, or approximately 6 cm proximal to the duodenum, and extending up to the Angle of His. This results in a small, banana-shaped stomach of roughly 100-200 ml volume. Typically up to five (5) “passes” or applications of a 60 mm stapler are needed in order to create the sleeve.

The gastrectomy can be visualized with the endoscope during the procedure, and the surgeon can see when the transected stomach, including the fundus and greater curvature of the stomach, is completely freed and then remove it from the peritoneum through the left flank port incision. The staple line along the remaining tubularized stomach is then typically over sewn and tested for any leaks through insufflations with the gastroscope, while the remnant stomach is submerged under irrigation fluid. The staple line is concurrently evaluated for bleeding both intraperitoneally with the laparoscope as well as intraluminally with the gastroscope. A drain is typically left in the left upper quadrant along the sleeve gastrectomy staple line.

Alternatively, access to the abdominal cavity for minimally invasive sleeve gastrectomy can be obtained using single incision laparoscopy surgery (SILS™). SILS™ allows certain highly trained surgeons to perform laparoscopic procedures such as gastrectomy and hysterectomy through the umbilicus, leaving no visible surgical scars. The concept of SILS™ is simple. Instead of using three or four separate ¼ to ½ inch abdominal incisions utilized in standard laparoscopic surgery (or the six-inch incision required by traditional surgery), SILS™ uses a single 2-4 cm (or 1 inch) incision in the umbilicus. Up to three instruments can be passed through this single port. This technique avoids multiple wounds and trocars but can be more challenging to technically manipulate tissue, due to the fact that all instruments are entering from a common site. The major advantage over traditional laparoscopic surgery is that there are no visible scars. The only scar is hidden within the umbilicus. Also, since there are fewer incisions, there is less tissue trauma and less risk of injury from multiple trocar insertions. SILS™ can be substituted for virtually any laparoscopic procedure.

A typical problem encountered during the gastrectomy procedure, no matter which surgical approach is used, arises when the sleeve is being created. As noted above, formation of the sleeve typically includes using a linear cutting stapler to serially staple and transect the stomach. Such endoscopic stapling devices have been developed for the specific needs of endoscopic and/or laparoscopic surgical procedures and are disclosed in, for example, U.S. Pat. No. 5,326,013 (Green, et al.); U.S. Pat. No. 5,332,142 (Robinson, et al.); U.S. Pat. No. 6,241,139 (Milliman et al.); and U.S. Pat. No. 7,967,180 (Scirica), the entire contents of each of which are incorporated herein by reference in their entirety.

Stapling typically begins at the antrum and proceeds in an upward direction towards the Angle of His, staying along a staple line that is just to the anatomic left and lateral of the endoscope. Each firing of the stapler, causing dividing and sealing of the stomach tissue, is a non-reversible step in the procedure, so each firing must be done with extreme precision and accuracy.

Since up to five passes of the stapler are typically needed in order to create the sleeve, the surgeon or assistant charged with retracting the large amount of stomach tissue outside of the sleeve area generally finds this tissue to be floppy and difficult to apply traction to. Also, due to the limited degree of motion available while working with an instrument positioned through a trocar port, it may be quite difficult for the surgeon to repeatedly manipulate a typical prior art grasping instrument within the peritoneum, in order to grasp and position the stomach tissue along the planned staple line. With multiple stapler passes needed to complete the sleeve, it can become very difficult to continually gather and apply traction to the large amount of stomach tissue that needs to be resected.

Thus, it can be appreciated that, during a laparoscopic sleeve gastrectomy procedure, the stapling process can be difficult to accomplish unless a constant, uniform traction can be applied along the staple line during the entire stapling process. If such tension could be maintained along the staple line during the entire serial stapling process, the staples could be placed more quickly, more efficiently, and in a substantially straight line to create a uniform sleeve from bottom to top.

In light of the above, it would be advantageous to provide a traction device and method for use during the stapling portion of a laparoscopic sleeve gastrectomy procedure in which there is no need to release and then re-grasp the stomach portion to be resected after each firing of the stapler. It would also be advantageous to provide a grasping instrument or traction device that can apply uniform traction to the stomach and provide proper tension, exposure and visualization of the staple line during a sleeve gastrectomy procedure. It would also be desirable to provide a device capable of maintaining uniform tension along the staple line for the entire stapling process. It would further be advantageous to provide a gastric traction device which can be coupled with a second surgical device, such as a serial stapler, during a sleeve gastrectomy procedure. Finally, it would be desirable to provide a device which would remedy the technical challenges of tissue exposure and manipulation presented by single incision laparoscopic surgery.

### SUMMARY OF THE INVENTION

The gastric traction device of the present invention allows the surgeon or assistant to gather and hold onto the large amount of stomach tissue that is typically resected during a laparoscopic sleeve gastrectomy procedure. The invention also relates to a method of using the traction device disclosed herein for grasping and manipulating the stomach and providing uniform tension at a consistent intensity during the stapling portion of the sleeve gastrectomy procedure.

A first aspect of the invention relates to a gastric traction device for providing uniform tension along a predetermined staple line for a sleeve gastrectomy procedure, the device comprising: (a) a proximal end comprising operation and control means, the operation and control means including a handle assembly for gripping and manipulating the device; (b) a central member coupled to the proximal end and sized to fit through a conventional trocar; and (c) a distal end coupled to the central member and adapted to be introduced through the trocar and into an operating field, the distal end comprising a clamp assembly including a first elongated jaw member and a second elongated jaw member, wherein each of the elongated jaw members are substantially equal to the length of the predetermined staple line, and wherein the elongated jaw members cooperate with one another to grasp the stomach tissue and apply uniform tension along their entire length.

A second aspect of the invention is a method for creating and maintaining uniform tension along a predetermined staple line during a sleeve gastrectomy procedure, comprising the steps of: (a) grasping the stomach tissue to be resected with a gastric traction device having a pair of elongated jaw members; (b) pulling the stomach tissue with the elongated jaw members in a direction that creates a uniform tension along the predetermined staple line; and (c) maintaining the uniform tension created at a consistent intensity along the staple line during the stapling portion of the sleeve gastrectomy procedure, whereby the staples can be quickly and easily placed in a straight line to create a uniform sleeve or pouch.

A third aspect of the invention is a device for use during a sleeve gastrectomy procedure, the device comprising: (a) a handle assembly for gripping and manipulating the device; (b) a central member extending distally from the handle assembly; and (c) a clamp assembly coupled to the central member at a proximal end, the clamp assembly comprising a first elongated jaw member and a second elongated jaw member, wherein each of the elongated jaw members are substantially equal to the length of a predetermined staple line, and wherein the elongated jaw members cooperate with one another to grasp the stomach tissue and apply uniform tension along their entire length.

While the nature and advantages of the present invention will be more fully appreciated from the following drawings and detailed description, showing the contemplated novel construction, combinations and elements as herein described, and more particularly defined by the appended claims, it is understood that changes in the precise embodiments of the present invention are meant to be included within the scope of the claims, except insofar as they may be precluded by the prior art.

### BRIEF DESCRIPTION OF THE DRAWINGS

The detailed description of the drawings particularly refers to the accompanying figures in which:

FIG. 1 is a perspective view of a stomach which schematically shows the staple line in relation to the stomach tissue for a sleeve gastrectomy procedure.

FIG. 2 is a side perspective view of a gastric traction device in accordance with an illustrative embodiment of the invention.

FIG. 3 is a perspective view of a gastric traction device including an articulating and rotating elongated shaft in accordance with an illustrative embodiment of the invention.

FIG. 4 is a perspective view of a gastric traction device including floating jaw members in accordance with another illustrative embodiment of the invention.

### DETAILED DESCRIPTION OF THE DRAWINGS

The embodiments of the invention described herein are not intended to be exhaustive or to limit the invention to precise forms disclosed. Rather, the embodiments elected for description have been chosen to enable one skilled in the art to practice the invention.

As used herein, when referring to the device of the present invention the term “proximal” will refer to the portion of the device closest to the operator and the term “distal” will refer to the portion of the device furthest from the operator.

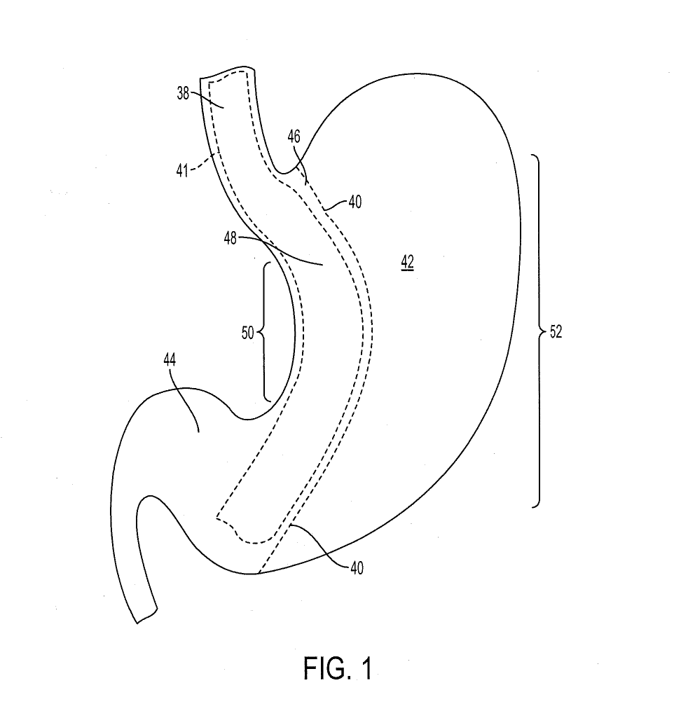

As noted above, the stapling procedure of a sleeve gastrectomy can be difficult to accomplish unless the staple line can be well-visualized and uniform tension can be applied and maintained along a predetermined staple line throughout the stapling process. Looking at FIG. 1, the staple line 40 typically extends from the area of the pylorus 44 at the distal end of the stomach 42 to the Angle of His 46 at the proximal end, which is typically between about 10 cm to about 18 cm, depending on the patient's stomach size. The surgeon typically uses an endoscope to place a size 10-14 mm bougie 41 down the esophagus 38 and inside the stomach 42 along the medial side, in the area of the lesser curvature 50. The size of the bougie 41 placed will typically define the size of the sleeve 48 created. The surgeon or an assistant then must grasp the portion of the stomach 42 to be resected, and then retract the stomach flat and laterally in the direction of the greater curvature 52. The staple line 40 is defined along the anatomic left side of the sleeve 48, and is typically located about 2-3 cm to the left of the edge of the lesser curvature 50 of the stomach.

Traction of the stomach tissue 42 away from the staple line 40 must be maintained while the surgeon uses a 3.5 or 4.8 mm linear stapler that typically cuts and then applies staples along the staple line 40. Because the portion of the stomach 42 to be resected is typically large, unwieldy and floppy, it can be difficult to maintain constant and uniform traction along the entire length of the staple line 40, and therefore it is possible that the stomach could fold upon itself and interrupt the surgeon's ability to accurately apply the stapler. The traction device and method of the present invention allows the surgeon or assistant to create and maintain uniform tension along the staple line 40 by providing the ability to grasp and pull the stomach tissue to be resected laterally, in a direction generally away from the staple line 40 and towards the greater curvature 52 of the stomach, and to maintain this uniform tension with a consistent force during the stapling procedure. In this manner, the staples can be quickly and easily placed in a straight line parallel to the left side of the bougie 41 to create a uniform sleeve or pouch.

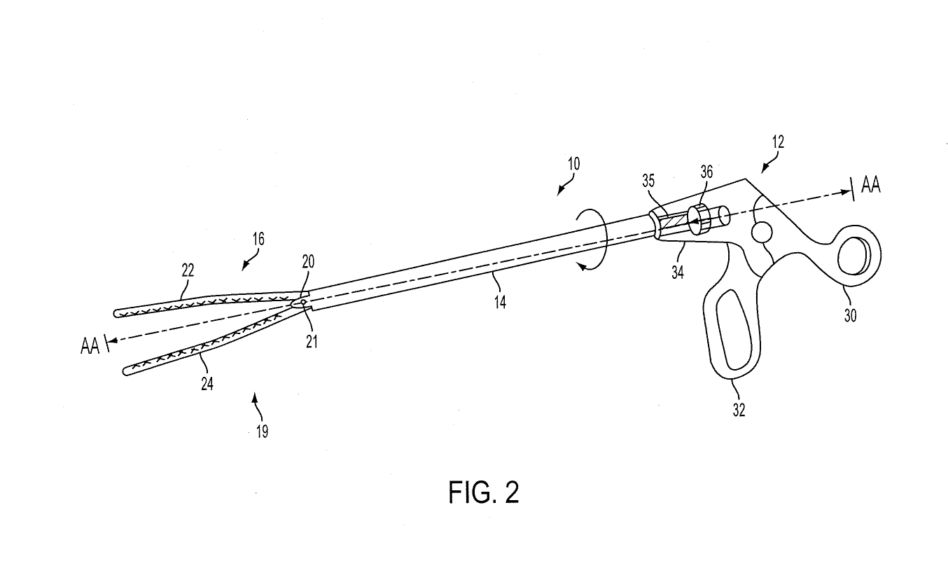

FIG. 2 illustrates one embodiment of the gastric traction device of the invention 10. The device 10 includes a proximal end 12 in the form of a handle assembly 15 comprising operation and control means for gripping and manipulating a distal end 16 which includes a clamp assembly 19 made of a pair of elongated, cooperating jaw members 22, 24. A central, endoscopic member 14 extends distally from the proximal end of handle assembly 15 to the distal end 16, and an articulating section 20 pivotally connects the elongated jaw members 22, 24 to the central member 14 by a pin 21. The proximal end 12 includes a stationary handle 30, a movable handle 32, and a barrel portion 34. The handle assembly 12 allows for opening and closure of the elongated jaw members 22, 24, and communicates with the distal end 16 via the central member 14. The handle assembly 12 includes means for moving the elongated jaw members 22, 24 from an open to a closed position. The elongated jaw members 22, 24, which are typically hinged together by the articulating section 20 at their proximal ends, allow for placement and locking of the device onto the stomach tissue.

The central, endoscopic member 14 also defines a longitudinal axis A-A along its length, and is sufficiently sized (typically about 5 mm in diameter) to position the distal end 16 (wherein the jaw members 22, 24 are in the closed position) through a trocar or cannula and into the operating field within the peritoneum. The central member 14 is typically rotatable around longitudinal axis A-A. For rotation, a cam member 35 can be mounted within the handle assembly 12 and connected to central member 14, such that when the cam member 35 is caused to rotate about longitudinal axis A-A, so does the central member 14. An articulation actuator 36 is supported on or within the handle 12 and operable to impart rotational motion to the cam member 35.

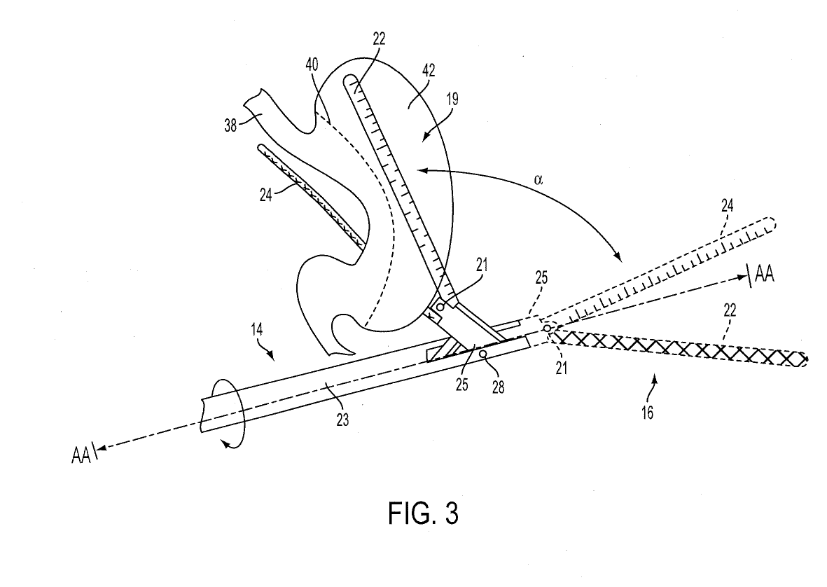

In another embodiment of the invention shown in FIG. 3, the central member 14 includes a first portion 23 extending from the handle, and a second portion 25 coupled to the first portion 23 and supporting the distal end 16. The first portion 23 defines a longitudinal axis A-A along its length, and is sufficiently sized to position the distal end 16 through a cannula. In addition to being rotatable as described for the device of FIG. 1, the distal end 16 of FIG. 3 is also articulatable relative to the longitudinal axis A-A defined by the first portion 23. A hinge or joint 28 established between the first and second portions 23, 25 of the central member 14 permits the second portion 25 and the cooperating elongated jaw members 22, 24 to articulate or pivot relative to the longitudinal axis A-A through an angle a, as shown.

Rotation about longitudinal axis A-A can be accomplished in the manner described for FIG. 2 above, wherein a cam member can be mounted within the handle and connected to the central member 14, and an articulation actuator causes the cam member to rotate. In the embodiment of FIG. 3, however, the cam member is configured to impart a force to both the first portion 23 and the second portion 25 of the central member 14 via the hinge 28 as the cam member rotates, to cause the second portion 25 to rotate and thus rotate the jaw members 22, 24 through angle a as well. A more detailed discussion of an instrument having an articulatable and rotatable end effector similar to the articulation and rotation of the distal end described above may be found in published U.S. Pat. App. No. 20100324551 to Gerhardt or U.S. Pat. No. 7,891,533 to Green et al., both of which are incorporated by reference herein in their entirety.

As illustrated in FIG. 3, the elongated jaw members 22, 24 of the invention are typically adapted to grasp the stomach tissue 42 and apply uniform tension along their entire length, and can include engaging surfaces that have a waffle-type pattern to aid in grasping the stomach tissue, without tearing or shredding the tissue. The elongated jaw members are typically 15 cm in length; however, they can be manufactured to be from about 10 cm to about 18 cm in length, more preferably from about 12 cm to about 15 cm in length. The length of each of the elongated jaw members 22, 24 is intended to substantially match the length of the predetermined staple line 40, which is typically from about 10 cm to about 18 cm in length and extends from about the pyloric area to about the area of the Angle of His. It is understood by a person of ordinary skill in the art that this length can vary from patient to patient, depending on the patient's stomach size and the size of the sleeve to be created by the surgeon.

The instrument of FIG. 3 allows the distal end 16 to be aligned with the longitudinal axis A-A of the instrument to facilitate insertion through a cannula, and thereafter the elongated cooperating jaw members 22, 24 can be caused to articulate as well as rotate in order to appropriately engage the stomach tissue. A flexible control wire may be provided to open or close the elongated jaw members 22, 24. The control wire may extend through an outer shaft from the handle 12 to the elongated jaw members such that the surgeon may create a tension in the control wire to cause the jaws to move closer to one another. The closure or clamping force generated in the elongated jaw members 22, 24 may be directly related to the tension in the control wire applied by the surgeon.

The gastric traction device 10 may or may not include a locking function, or the locking function may be present but is not used by the surgeon, depending on user preference. Some users may find it easier to create uniform tension along the staple line by continuing to clamp the elongated jaw members closed by hand, while others may prefer to lock the jaw members while retracting the stomach tissue. The device, including the proximal end 12, central member 14, and distal end 16, can be formed of a thermoplastic material, e.g., polycarbonate. Alternately, other materials having the requisite strength requirements may be used to form the device, e.g., surgical grade metals. The inventive device is typically a reusable device, but it could be manufactured to be disposable as well.

The elongated jaw members 22, 24 cooperate with one another to grasp the stomach tissue and apply uniform tension along the entire length of the jaw members. As noted above, a locking function can be included within the handle portion to reversibly couple or secure the first jaw member 22 and the second jaw member 24 into a clamped or approximated position. In addition, the first jaw member 22 can be movable in relation to the second jaw member 24 to move the clamp assembly 19 between an open and clamped position. The clamp assembly 19 may alternatively be arranged such that the second jaw member 24 is movable in relation to the first jaw member 22, or both jaw members 22,24 may be movable. As further detailed herein, an illustrative clamp assembly 119 may include floating jaw members 122, 124 to assist in maintaining equal force along their full lengths.

As illustrated in FIG. 3, the elongated jaw members 22, 24 can include a ruler or measuring scale, so that the surgeon can visually estimate the length of the staple line, stomach tissue to be resected, etc. Since the typical length of a stapler used for the procedure is between about 3 cm to about 5 cm, with a common length being 4.5 cm, unless there is some means to continuously retract the entire amount of stomach tissue that is outside of the sleeve area during stapling, the stomach tissue to be resected can become difficult to manage during the stapling procedure. The gastric traction device and method of the invention solves this problem. The device also allows the surgeon to flatten and retract the stomach tissue laterally throughout the stapling procedure, without having to release and then re-grasp the stomach portion to be resected after each firing of the stapler. The clamp assembly 19 creates uniform tension along the entire length of its jaw members 22, 24, and thus the staple line, so that the proper tension, exposure and visualization of the staple line is available during the entire stapling process. The device and method of the invention can therefore alleviate the technical challenges of tissue exposure and manipulation presented by both conventional laparoscopic surgery and single incision laparoscopic surgery.

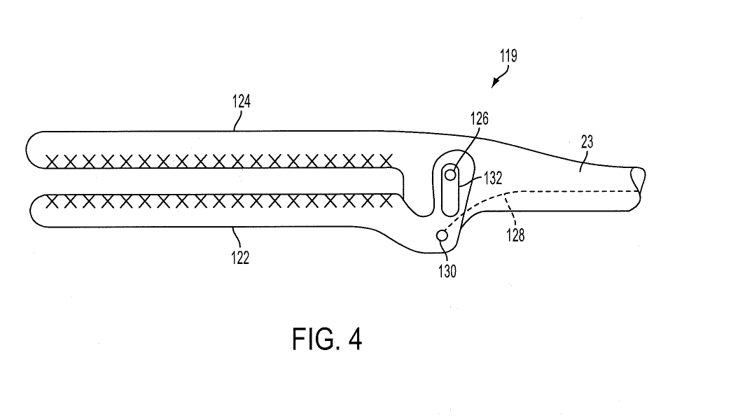

With reference to FIG. 4, floating jaw members 122, 124 may be utilized in a further illustrative clamp assembly 119. The floating jaw members 122, 124 assist in maintaining equal force along their full lengths. Moveable jaw member 122 may be pivotally connected at a pin 126 to stationary jaw member 124 and handle assembly 12. An actuator, such as a flexible control wire 128 may be provided to open and close jaw members 122, 124. More particularly, control wire 128 may be operably coupled to moveable jaw member 122 at pin 130, which is laterally offset from pivot pin 126. Moveable jaw member 122 may be laterally adjusted relative to pivot pin 126 and stationary jaw member 124 to control clamping force. Illustratively, moveable jaw member 122 includes a slot 132 receiving pin 126.

The device of the invention typically varies in diameter from 1.8 mm to 15 mm, but is typically designed to pass through a 5-10 mm cannula. The instrument can be generally made of different lengths, but typically the length of the instrument varies from 18 to 45 cm, and preferably about 36 cm in length for use with adults, and about 28 cm in pediatric practice. Shorter instruments 18 to 25 cm are adapted for cervical and pediatric surgery. Certain procedures for adults can also be performed with shorter instruments where the space is constricted. 45 cm instruments are used in obese or very tall patients. For better ergonomics, half of the central member typically lies inside the abdomen, and half outside the abdomen, in order to stabilize the port and allow the surgery to be more expedient.

In use, the central endoscopic member 14 is inserted through the cannula and into the body, preferably through an endoscopic tube. A single site or port may be used for the gastric traction device 10 and other instruments and/or tools. More particularly, the device 10 may be placed in a separate trocar, a single site surgical procedure or an open laparotomy. It is further preferred that the endoscopic tube apparatus be capable of maintaining a sealed pneumoperitoneum, with the internal sealing member of the housing further maintaining this seal despite introduction of the instrument into the endoscopic tube. The elongated jaw members 22, 24 are then placed around the stomach 42, directly adjacent and parallel to the staple line 40 (See FIG. 1). More specifically, the elongated jaw members 22, 24 can grasp the stomach tissue to be resected at a location about 0.1 centimeters to about 0.5 centimeters to the anatomic left of the staple line, and then used to pull or retract the stomach tissue flat and laterally, away from the staple line and towards the greater curvature of the stomach, with uniform tension. The clamp assembly 19 including the elongated cooperating jaw members 22, 24 can then be locked onto the stomach via a locking function, if desired, or the surgeon can merely continue to create tension by hand (or via a control wire) to cause the jaws to remain clamped. As the elongated jaw members 22, 24 are manipulated by the surgeon via the proximal handle assembly 12 to pull the portion of stomach to be resected to the side, the lateral traction provides uniform tension along the length of the staple line due to the uniform clamping of the jaw members 22, 24 along the length of the staple line. Therefore, the staples can be placed in a straight line parallel to the staple line and the bougie 41, to create a uniform sleeve from bottom to top.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative system and method, and illustrated examples shown and described. Accordingly, departures may be made from such details without departing from the scope or spirit of the invention.

1. A gastric traction device for providing uniform tension along a predetermined staple line for a sleeve gastrectomy procedure, the device comprising:
a) a proximal end comprising operation and control means, the operation and control means including a handle assembly for gripping and manipulating the device;
b) a central member coupled to the proximal end and sized to fit through a conventional trocar; and
c) a distal end coupled to the central member and adapted to be introduced through the trocar and into an operating field, the distal end comprising a clamp assembly including a first elongated jaw member and a second elongated jaw member, wherein each of the elongated jaw members are substantially equal to the length of the predetermined staple line, and wherein the elongated jaw members cooperate with one another to grasp the stomach tissue and apply uniform tension along their entire length.

2. The method of claim 1, wherein the predetermined staple line extends from about the pyloric area to about the area of the Angle of His.

3. The device of claim 1, wherein the elongated jaw members are each about 10 cm to about 18 cm in length, and more preferably about 12 cm to about 15 cm in length.

4. The device of claim 1, wherein the elongated jaw members are each 15 cm in length.

5. The device of claim 1, wherein the elongated jaw members include engaging surfaces that have a waffle-type pattern to aid in grasping the stomach tissue, without tearing or shredding the tissue.

6. The device of claim 1, wherein at least one of the elongated jaw members includes a ruler or measuring scale, so that the surgeon can visually estimate the length of the staple line and the stomach tissue to be resected.

7. The device of claim 1, wherein the proximal end imparts rotating motion to the clamp assembly, the proximal end including a cam member rotatably mounted about the longitudinal axis of the central member and configured to impart a force to the clamp assembly upon rotation of the cam member to effect pivotal motion of the elongated jaw members.

8. The device of claim 1, wherein the central member and the distal end are sized to fit through a port that is used in conjunction with a single-incision laparoscopic surgery (SILS™) procedure.

9. The device of claim 7, wherein the proximal end imparts articulating motion to the clamp assembly, the central member including a first portion extending from the proximal end, a second portion supporting the distal end, and a joint connecting the first and second portions, wherein the joint permits the second portion of the central member and the distal end to articulate or pivot relative to the longitudinal axis of the central member.

10. A method for creating and maintaining uniform tension along a predetermined staple line during a sleeve gastrectomy procedure, comprising the steps of:
a) grasping the stomach tissue to be resected with a gastric traction device having a pair of elongated jaw members;
b) pulling the stomach tissue with the elongated jaw members in a direction that creates a uniform tension along the predetermined staple line; and
c) maintaining the uniform tension created at a consistent intensity along the staple line during the stapling portion of the sleeve gastrectomy procedure, whereby the staples can be quickly and easily placed in a straight line to create a uniform sleeve or pouch.

11. The method of claim 11, wherein the predetermined staple line extends from about the pyloric area to about the area of the Angle of His.

12. The method of claim 11, wherein the stomach tissue to be resected is pulled in a direction generally laterally and away from the staple line towards the greater curvature of the stomach.

13. The method of claim 11, wherein the steps are performed using a gastric traction device, the device comprising:
i) a proximal end comprising operation and control means, the operation and control means including a handle assembly for gripping and manipulating the device;
ii) a central member coupled to the proximal end and sized to fit through a conventional trocar; and
iii) a distal end coupled to the central member and adapted to be introduced through the trocar and into an operating field, the distal end comprising a clamp assembly including a first elongated jaw member and a second elongated jaw member, wherein the elongated jaw members cooperate with one another to grasp the stomach tissue and apply uniform tension along their entire length.

14. The method of claim 12, wherein the elongated jaw members are each 15 cm in length.

15. The method of claim 14, wherein the elongated jaw members are placed from about 0.1 centimeters to about 0.5 centimeters lateral to the staple line.

16. A device for creating a uniform sleeve during a sleeve gastrectomy procedure, the device comprising:
a) a handle assembly for gripping and manipulating the device;
b) a central member extending distally from the handle assembly; and
c) a clamp assembly coupled to the central member at a proximal end, the clamp assembly comprising a first elongated jaw member and a second elongated jaw member, wherein each of the elongated jaw members are substantially equal to the length of a predetermined staple line, and wherein the elongated jaw members cooperate with one another to grasp the stomach tissue and apply uniform tension along their entire length.
